(19) United States
(12) Patent Application Publication
(10) Pub. No.: US 2006/0286172 A1
(43) Pub. Date: Dec. 21, 2006

(54) Pharmaceutical compositions comprising prostanoid-receptor agonists and methods of making and using the same

(76) Inventor: Anu Mahashabde, Kendall Park, NJ (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/446,525

(22) Filed: Jun. 5, 2006

Related U.S. Application Data
(60) Provisional application No. 60/686,973, filed on Jun. 3, 2005.

(51) Int. Cl. A61K 9/14

(52) U.S. Cl. 424/488

(57) ABSTRACT

The present invention is related to pharmaceutical compositions comprising prostanoid-receptor agonists, intravaginal dosage forms comprising the same, and methods of making and using the same. The present invention is also related to a controlled release pharmaceutical gel for vaginal administration, the pharmaceutical gel comprising: (a) misoprostol; (b) a cellulose derivative; and (c) a polyol; wherein the gel is a substantially nonaqueous gel, and wherein the gel forms a hydrogel when placed in a vaginal tract.

This application claims the benefit of the filing date of U.S. Application No. 60/686,973, filed Jun. 3, 2005, which is incorporated by reference herein in its entirety.

### BACKGROUND OF THE INVENTION

1. Field of the Invention

The present invention is related to pharmaceutical compositions comprising prostanoid-receptor agonists, intravaginal dosage forms comprising the same, and methods of making and using the same. The present invention is also related to a controlled release pharmaceutical gel for vaginal administration, the pharmaceutical gel comprising: (a) misoprostol; (b) a cellulose derivative; and (c) a polyol; wherein the gel is a substantially nonaqueous gel, and wherein the gel forms a hydrogel when placed in a vaginal tract.

2. Background Art

Prostanoids are members of the eicosanoid family of phospholipid mediators, and include prostaglandins and thromboxanes. Numerous prostaglandins and thromboxanes are known in the art. Typically, these compounds function as agonists for prostanoid receptors, e.g., seven-transmembrane G-protein-coupled receptors, to elicit their distinct biological effects.

Prostaglandins are a group of biologically active compounds derived from 20 carbon polyunsaturated fatty acids. They are classified based upon the specific structure of the pentane ring of the core prostaglandin chemical structure, for example, prostaglandin A, prostaglandin B, prostaglandin D, prostaglandin E, prostaglandin F, and prostaglandin I. The distinct biological effects of the prostaglandin classes are due in part to the mediation of their activity by prostanoid receptors which are expressed in a tissue-specific manner and which specifically recognize the different classes of prostaglandins. Prostaglandins of the E and F classes (PGE and PGF) are of particular interest due to their ability to exert biological effects on tissues of the cervix and myometrium. PGE and PGF class members are generally represented by the following chemical structures:

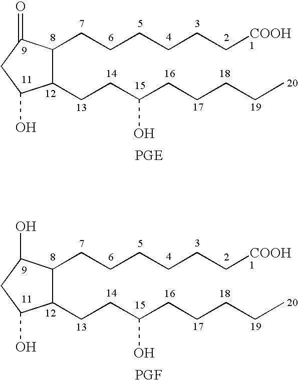

Due to their uterotropic effects, PGE and PGF have been used clinically for, inter alia, the induction of labor, the induction of cervical ripening, and for the termination of pregnancy. However, use of primary prostaglandins of the E and F groups (PGE₁, PGE₂, and PGF_{2α}) has two principal disadvantages: PGE₁, PGE₂, and PGF₂, have relatively short half-lives and their use can result in gastrointestinal side effects. To overcome the chemical instability of these compounds, chemical derivatives of PGE and PGF have been developed which are not substrates for the initial steps of enzymatic prostaglandin degradation and thus have increased metabolic stability. These chemical derivatives include, but are not limited to: PGE₁ derivatives, such as misoprostol, limaprost, and gemeprost; PGE₂ derivatives, such as dinoprostone and sulprostone; and PGF_{2α} derivatives, such as carboprost.

These PGE and PGF derivatives have also been used to induce both labor and cervical ripening, to expediate the completion pregnancy, and to cause medical abortions. However, the systemic delivery of these derivatives can result in side effects, e.g., gastrointestinal problems. Additionally, local delivery may result in inconsistent bioavailability and difficulty in dose management.

There exists a need in the art to provide local intravaginal delivery of prostanoid-receptor agonists, such as members of the PGE and PGF classes, to avoid complications associated with systemic administration and whereby active agent delivery can be controlled such that the release of the prostanoid-receptor agonist is provided at a specific rate of release over a desired period of time.

### BRIEF SUMMARY OF THE INVENTION

The present invention is directed to a controlled release pharmaceutical gel for vaginal administration, the pharmaceutical gel comprising: (a) misoprostol; (b) a cellulose derivative; and (c) a polyol; wherein the gel is a substantially nonaqueous gel, and wherein the gel forms a hydrogel when placed in a vaginal tract.

In some embodiments, the pharmaceutical gel further comprises (d) a mucoadhesive agent. Various mucoadhesives can be used. In some embodiments, the mucoadhesive agent is selected from the group consisting of a cross-linked acrylic acid-based polymer, polycarbophil, chitosan, polyethylene oxide, and combinations thereof. In some embodiments, the mucoadhesive agent is a cross-linked acrylic acid-based polymer.

Various cellulose derivatives can be used. In some embodiments, the cellulose derivative is selected from the group consisting of methyl cellulose, hydroxyethyl cellulose, hydroxypropyl cellulose, hydroxypropyl methylcellulose, and combinations thereof. The cellulose derivative can be hydroxyethyl cellulose having a molecular weight from about 50,000 MW to about 2,000,000 MW. In some embodiments of the present invention, about 75% (w/w) or greater of the cellulose derivative is about 80,000 MW to about 100,000 MW and about 25% (w/w) or less of the cellulose derivative is about 800,000 MW to about 1,200,000 MW. Alternatively, about 50% (w/w) of the cellulose derivative is about 80,000 MW to about 100,000 MW and about 50% (w/w) of the cellulose derivative is about 800,000 MW to about 1,200,000 MW. In other embodiments of the present invention, about 20% (w/w) or less of the cellulose derivative is about 80,000 MW to about 100,000 MW and about 80% (w/w) or greater of the cellulose derivative is about 800,000 MW to about 1,200,000 MW.

Various ratios of cellulose derivative to polyol can be used. For example, the ratio of the cellulose derivative to polyol can be about 1:2 to about 1:10 (w/w), or about 1:4 to about 1:7 (w/w).

In the present invention, the polyol can be selected from the group consisting of glycerin, propylene glycol, polyethylene glycol, or combinations thereof. In some embodiments, the polyol is glycerin.

In some embodiments, the pharmaceutical gel of the present invention further comprises a pharmaceutically acceptable additive, pharmaceutically acceptable stabilizing agent, or combination thereof. Pharmaceutically acceptable additives can be selected from the group consisting of, but not limited to, polaxomers, carbomers, polyvinyl alcohol, silicon dioxide, sodium carboxymethyl cellulose and combinations thereof. In the present invention, the pharmaceutically acceptable stabilizing agent can be selected from the group consisting of, but not limited to α-lipoic acid, α-tocopherol, ascorbyl palmitate, benzyl alcohol, biotin, bisulfites, boron, butylated hydroxyanisole, butylated hydroxytoluene, ascorbic acid, carotenoids, calcium citrate, acetyl-L-carnitine, chelating agents, chondroitin, chromium, citric acid, coenzyme Q-10, cysteine, cysteine hydrochloride, 3-dehydroshikimic acid, EDTA, ferrous sulfate, folic acid, fumaric acid, alkyl gallates, garlic, glucosamine, grape seed extract, gugul, magnesium, malic acid, metabisulfite, N-acetyl cysteine, niacin, nicotinomide, nettle root, ornithine, propyl gallate, pycnogenol, saw palmetto, selenium, sodium bisulfite, sodium metabisulfite, sodium sulfite, potassium sulfite, tartaric acid, thiosulfates, thioglycerol, thiosorbitol, tocopherol, tocopherol acetate, tocopherol succinate, tocotrienal, d-α-tocopherol acetate, vitamin A, vitamin B, vitamin C, vitamin D, vitamin E, zinc, and combinations thereof.

The present invention can further comprise a supportive device. In some embodiments, the supportive device is annular. In some embodiments, the supportive device can comprise a cavity sufficient for retaining the pharmaceutical gel.

The present invention can also be directed to an intravaginal dosage form comprising the pharmaceutical gel of the present invention, wherein the dosage form is selected from the group consisting of a vaginal ring, a soft tablet, a capsule, a vaginal bead, and a vaginal suppository. In some embodiments, the intravaginal dosage form further comprises a withdrawal cord. In some embodiments, the dosage form is a vaginal ring. The vaginal ring can comprise a cavity sufficient for retaining the pharmaceutical gel. In some embodiments, the vaginal ring is suitable for immediate release of the pharmaceutical gel. In some embodiments, the vaginal ring is suitable for controlled release of the pharmaceutical gel.

The intravaginal dosage form can release misoprostol at various rates. In some embodiments, the vaginal ring has a release rate of about 2 μg/hour to about 100 μg/hour of misoprostol, about 2 μg/hour to about 50 μg/hour of misoprostol, or about 2 μg/hour to about 20 μg/hour of misoprostol. In some embodiments, the misoprostol is released for about 1 hour to about 24 hours.

The present invention is also directed to a pharmaceutical gel comprising: (a) misoprostol; (b) two or more cellulose derivatives; and (c) a polyol; and (d) a mucoadhesive agent; wherein the gel is substantially nonaqueous gel, and wherein gel forms a hydrogel when placed in a vaginal tract.

The present invention is also directed to a method of inducing uterine contractions in a female, the method comprising locally administering to the vagina of the female a therapeutically effective amount of the pharmaceutical gel of the present invention. In some embodiments, the local administration to the vagina is achieved by using an intravaginal applicator. In some embodiments, the method comprises locally administering to a vagina of the female a therapeutically effective amount of the pharmaceutical gel of the present invention. The local administration can be achieved in numerous ways, e.g., the local administration to the vagina can be achieved by using a vaginal ring, a soft tablet, a capsule, a vaginal bead, or a vaginal suppository.

The present invention is also directed to a method of inducing cervical dilatation in a female, the method comprising locally administering to the vagina of the female a therapeutically effective amount of the pharmaceutical gel of the present invention. The local administration can be achieved in numerous ways, e.g., the local administration to the vagina can be achieved by using a vaginal ring, a soft tablet, a capsule, a vaginal bead, or a vaginal suppository.

### BRIEF DESCRIPTION OF THE FIGURES

FIG. 1 provides in vitro release curves for two vaginal rings containing 25 μg and 50 μg, respectively, of misoprostol delivered over a 4 hour period of time. Formulation Rₓ1 (-▾-) displayed a release rate of 6.4 μg/hr for the 25 μg dose and Formulation Rₓ2 (-▪-) displayed a release rate of 12.0 μg/hr for the 50 μg dose.

FIG. 2 provides in vitro release curves for three vaginal rings containing 50 μg misoprostol delivered over a 24 hour period of time. Formulation 1 (-▪-) included 50 μg misoprostol mixed with a 1:4 ratio of HEC (Natrosol® 250M) to glycerin and displayed a release rate of 4.7 μg/hour. Formulation 2 (-▾-) included 50 μg misoprostol mixed with a 1:5 ratio of HEC (Natrosol® 250HX) to glycerin and displayed a release rate of 4.0 μg/hour. Formulation 3 (-●-) included 50 μg misoprostol mixed with a 1:5 ratio of HEC (80% Natrosol® 250HX/20% Natrosol® 250L) to glycerin and displayed a release rate of 4.6 μg/hour.

FIG. 3 provides in vitro release curves for three vaginal rings containing misoprostol delivered over a 4 hour period of time. Formulation 1 (Low Dose) (-●-) included 12.5 μg misoprostol mixed with a 1:6 ratio of HEC (75% Natrosol® 250L/25% Natrosol® 250HX) to glycerin and displayed an overall release rate of 4.16 μg/hour. Formulation 2 (Medium Dose) (-▪-) included 25 μg misoprostol mixed with a 1:6 ratio of HEC (75% Natrosol® 250L/25% Natrosol® 250HX) to glycerin and displayed an overall release rate of 6.25 μg/hour. Formulation 3 (High Dose) (-▾-) included 50 μg misoprostol mixed with a 1:6 ratio of HEC (75% Natrosol® 250L/25% Natrosol® 250HX) to glycerin and displayed an overall release rate of 12.5 μg/hour.

FIG. 4 provides in vitro release curve for vaginal rings containing 60 μg misoprostol dispersed in a 1:6 HEC (75% Natrosol® 250L/25% Natrosol® 250HX) to propylene glycol (Spectrum, Inc., USA) gel matrix delivered over a 4 hour period of time.

FIG. 5 provides in vitro release curve for vaginal rings containing 60 μg misoprostol dispersed in a 1:6 HEC (75% Natrosol® 250L/25% Natrosol® 250HX) to propylene glycol (Spectrum, Inc., USA)+PEG-400 30% (Spectrum, Inc., USA)+glycerin (EM Science, USA) gel matrix delivered over a 4 hour period of time.

FIG. 6 provides in vitro release curve for vaginal rings containing 60 μg misoprostol dispersed in a 1:6 HEC (75% Natrosol® 250L/25% Natrosol® 250HX) to propylene glycol (Spectrum, Inc., USA)+1% glycerin (EM Science, USA) gel matrix delivered over a 4 hour period of time.

### DETAILED DESCRIPTION OF THE INVENTION

The present invention is directed to a controlled release pharmaceutical gel for vaginal administration, the pharmaceutical gel comprising: (a) misoprostol; (b) a cellulose derivative; and (c) a polyol; wherein the gel is a substantially nonaqueous gel, and wherein the gel forms a hydrogel when placed in a vaginal tract.

The present invention is also directed to a pharmaceutical gel comprising a prostanoid-receptor agonist, one or more pharmaceutically acceptable cellulose derivatives, and a pharmaceutically acceptable polyol, wherein the ratio of the cellulose derivative to the polyol is about 1:2 to about 1:10 (w/w). The ratio of cellulose derivative to polyol can control the release of the prostanoid-receptor agonist for a desired period of time because the release mechanism of the prostanoid-receptor agonist is triggered upon solvation and is mediated by the swelling and/or dissolution of the gel matrix which controls the diffusion of the prostanoid-receptor agonist from the cellulose derivative-polyol matrix into the local vaginal environment.

As used herein, the phrase “pharmaceutical gel” refers to those compounds, materials, compositions, and/or dosage forms which form a gel and which are, within the scope of sound medical judgment, suitable for contact with the tissues of human beings and animals without excessive toxicity, irritation, allergic response, or other complications commensurate with a reasonable benefit/risk ratio.

The term “gel” refers to any semisolid system consisting of a suspension made up of a complex lattice matrix of large organic molecules interpenetrated by a liquid. The term “substantially nonaqueous gel” refers to a gel wherein the liquid present in the gel is predominantly not water. However, in a substantially nonaqueous gel, small amounts of water may be present, e.g., from the residue of the manufacturing process, from the storage environment, or from the atmosphere. The term “hydrogel” refers to a gel which absorbs water when in the presence of water. Thus, a hydrogel consists of a suspension made up of a complex lattice matrix of large organic molecules interpenetrated predominantly by water.

The term “polyol” refers to a compound having two or more hydroxyl radicals. Examples of suitable polyols include, but are not limited to, glycerin, ethylene glycol, propylene glycol, polyethylene glycol (PEG) (e.g., liquid-grade PEG), polyoxyethylene sorbitan fatty acid esters (e.g., polyoxyethylene 20 sorbitan monooleate), hydroxylated solvents having two or more hydroxyl groups, and combinations thereof. In some aspects of the invention, the polyol is glycerin.

“Controlled release pharmaceutical gel” refers to a pharmaceutical gel that can be administered to a patient to elicit a pharmaceutical effect such that the active agent is released at a specific release rate for a specific amount of time.

As used herein, the phrase “prostanoid-receptor agonist” refers to any member of the eicosanoid family of phospholipid mediators that binds to the prostanoid receptor and activates the cyclooxygenase pathway. Prostanoid-receptor agonists can possess antagonistic activity, in addition to agonistic activity, even within the same target tissue. Examples of suitable prostanoid-receptor agonists include, but are not limited to, prostaglandins, such as those of the prostaglandin E class (PGE) and prostaglandin F class (PGF), and thromboxanes. Suitable members of the PGE class include, but are not limited to, PGE₁ and PGE₂. Additionally, suitable members of the PGF class include, but are not limited to, PGF_{1α} and PGF_{2α}. The term “prostanoid-receptor agonist” also refers to any chemical compound that is a structural derivative or analogue of a prostaglandin or thromboxane. Such prostanoid-receptor agonists are known in the art and include, but are not limited to, prostaglandin E1 (PGE₁) derivatives such as limaprost, gemeprost, and misoprostol. Additional prostanoid-receptor agonists which are suitable for use in the present invention include, but are not limited to, prostaglandin E2 (PGE₂) derivatives such as dinoprostone and sulprostone. Additional prostanoid-receptor agonists which are also suitable for use in the present invention include, but are not limited to, prostaglandin F_{2α} (PGF_{2α}) derivatives such as carboprost.

As used herein, “misoprostol” refers to the synthetic prostaglandin E₁ chemical derivative, which is (±) methyl 11 (α), 16-dihydroxy-16-methyl-9-oxoprost-13E-en-1-oate. Misoprostol contains approximately equal amounts of two diastereomers, each being a racemic mixture of two optical isomers. The four isomers are SC-30422, SC-30423, SC-30248, and SC-30249, with isomer SC-30429 being the primary active isomeric form. Therefore, when isomer SC-30249 is used as the active ingredient, a dosage that is approximately ¼ the dosage of misoprostol is needed. The term “misoprostol” also refers to acids and salts of misoprostol.

As used herein, “about” refers to plus or minus 10% of the indicated number. For example, “about 1:3” indicates a ratio of 1:2.7 to 1:3.3.

As used herein, the “molecular weight” of a cellulose derivative refers to the molecular weight value as estimated from the apparent viscosity of the cellulose derivative.

The pharmaceutical gel of the present invention additionally comprises one or more pharmaceutically acceptable celluloses or cellulose derivatives. As used herein, a “cellulose derivative” refers to a modified cellulose polymer. The term cellulose derivative includes the various molecular weight species of the specific cellulose derivative used in the pharmaceutical gel of the present invention. Examples of suitable cellulose derivatives for use in the pharmaceutical gel of the present invention include, but are not limited to, the varying molecular weight species of methyl cellulose, hydroxyethyl cellulose, hydroxypropyl cellulose, hydroxypropyl methylcellulose, celluloses (e.g., microcrystalline cellulose and powdered cellulose), and combinations thereof. In some aspects of the invention, the cellulose derivative is a molecular weight species of hydroxyethyl cellulose, hydroxypropyl cellulose, hydroxypropyl methylcellulose, and combinations thereof. In other aspects, the cellulose derivative component of the gel can be a molecular weight grade of hydroxyethyl cellulose ranging from about 50,000 MW to about 2,000,000 MW, from about 90,000 MW to about 1,300,000 MW, or from about 100,000 MW to about 1,000,000 MW. In still other aspects, the cellulose derivative can be, but is not limited to, a hydroxyethyl cellulose having a molecular weight of about 90,000 MW, about 720,000 MW, about 1,000,000 MW, or about 1,300,000 MW, e.g., Natrosol® (Hercules, Inc., Wilmington, Del.) 250L, 250M, 250HX, and 250HXX, respectively.

The pharmaceutical gel of the present invention can also comprise two or more celluloses or cellulose derivatives in the same composition. In some aspects of the invention, the cellulose derivative component of the gel comprises multiple molecular weight species of methyl cellulose, hydroxyethyl cellulose, hydroxypropyl cellulose, hydroxypropyl methylcellulose, celluloses (e.g., microcrystalline cellulose and powdered cellulose), and combinations thereof. In some aspects, the cellulose derivative component of the gel comprises multiple molecular weight grades of hydroxyethyl cellulose, each cellulose derivative ranging from about 50,000 MW to about 2,000,000 MW, from about 90,000 MW to about 1,300,000 MW, or from about 100,000 MW to about 1,000,000 MW. In other aspects, the cellulose derivative component of the gel comprises multiple molecular weight grades of hydroxyethyl cellulose, with one or more hydroxyethylcellulose having a molecular weight of about 90,000 MW, about 720,000 MW, about 1,000,000 MW, or about 1,300,000 MW. In some embodiments, the cellulose derivative is from about 20%:80% of about 90,000 MW: about 1,000,000 MW, to about 80%:20% of about 90,000 MW: about 1,000,000 MW. In some embodiments, about 75% (w/w) or greater of the cellulose derivative is about 80,000 MW to about 100,000 MW and about 25% (w/w) or less of the cellulose derivative is about 800,000 MW to about 1,200,000 MW. In some embodiments, about 50% (w/w) of the cellulose derivative is about 80,000 MW to about 100,000 MW and about 50% (w/w) of the cellulose derivative is about 800,000 MW to about 1,200,000 MW. In some embodiments, about 20% (w/w) or less of the cellulose derivative is about 80,000 MW to about 100,000 MW and about 80% (w/w) or greater of the cellulose derivative is about 800,000 MW to about 1,200,000 MW.

To accomplish the desired formulation, the cellulose derivative and polyol are combined in a predetermined ratio, with or without additives, and mixed to form a gel matrix. While not being bound by a particular theory, the gelation of the components can result in increased hydrogen bonding interactions and in some aspects can be accelerated by heat curing. In some aspects of the invention, the prostanoid-receptor agonist is added to the premixed gel. In other aspects, the prostanoid-receptor agonist is mixed in situ with the other components of the formulation.

An advantage of the present invention lies in the ability to control the release of the prostanoid-receptor agonist over a desired period of time. Particularly advantageous is the zero order release kinetics that can be achieved by the pharmaceutical gel of the present invention. This property allows a provider to locally administer the pharmaceutical gel and achieve a specific release profile that is optimal for the desired effect, for example, the induction of uterine contractions in a female. The desired release rate of the pharmaceutical gel is achieved by the maintenance of the ratio of the cellulose derivative to the polyol at about 1:2 to about 1:10 (w/w). In some aspects of the invention, the ratio of the cellulose derivative to the polyol is about 1:4 to about 1:7 (w/w). If a slower release rate for the prostanoid-receptor agonist is desired, the amount of the cellulose derivative to the polyol can be about 1:2 (w/w) to about 1:5 (w/w). If a faster release rate is desired, the amount of the cellulose derivative to the polyol can be greater than 1:5 (w/w), with a decreasing amount of cellulose derivative relative to the polyol resulting in a faster release of the prostanoid-receptor agonist.

Thus, the present invention provides substantial advantages over other dosage forms in that the release rate of a prostanoid-receptor agonist from the pharmaceutical gel is easily adapted to provide for either the immediate release of the active agent, as in the case of controlling and/or reducing vaginal bleeding in emergency situations, or to provide for the controlled release of the active agent, as in the case of inducing cervical ripening. As such, the pharmaceutical gel of present invention possesses the flexibility to administer a prostanoid-receptor agonist over a broad range of release rates, from immediate release to controlled release and any combination thereof, by altering the ratio of the cellulose derivative component to the polyol component of the pharmaceutical gel.

The present invention can further comprise a “mucoadhesive agent” or “mucoadhesive.” “Mucoadhesive agent,” as used herein, refers to a substance, or mixture of substances, that is added to the pharmaceutical gel of the present invention to increase the extent of absorption to the site of administration, whereby the residence time of the gel at the site of absorption is prolonged, thereby increasing the period over which the active agent can be absorbed. Such agents also aid in increasing the intimate contact between the pharmaceutical dosage form and the absorbing tissue and lead to an increase in local active agent concentration and hence a higher active agent flux through the absorbing tissue. As a consequence, a constant level of the therapeutic moiety can be achieved with less frequent dosing. Examples of suitable mucoadhesive agents for use in the pharmaceutical gel include, but are not limited to, cross-linked acrylic acid-based polymer, polycarbophil, chitosan, polyethylene oxide, and combinations thereof. In some aspects of the invention, the mucoadhesive agent is a cross-linked acrylic acid-based polymer, such as Carbopol® (BF Goodrich, Inc., Cleveland, Ohio). Thus, in some embodiments the present invention can be directed to a pharmaceutical gel comprising: (a) misoprostol; (b) two or more cellulose derivatives; (c) a polyol; and (d) a mucoadhesive agent; wherein the gel is a substantially nonaqueous gel, and wherein the gel forms a hydrogel when placed in a vaginal tract.

The pharmaceutical gel of the present invention can further comprise a pharmaceutically acceptable excipient. As used here in, “excipient” refers to a substance, or mixture of substances, that is added to the pharmaceutical gel to give additional desired physical characteristics to the formulation. Examples of pharmaceutically acceptable excipients include, but are not limited to, pharmaceutically acceptable additives, pharmaceutically acceptable stabilizing agents, and combinations thereof.

The term “additive” as used herein refers to a substance, or mixture of substances, that is added to the pharmaceutical gel to give additional physical characteristics to the formulation such as increased viscosity, mass, or volume. Examples of suitable additives for use in the pharmaceutical gel include, but are not limited to, polaxomers (e.g., CAS no. 9003-11-6), carbomers (e.g. Carbopol®), polyvinyl alcohol, silicon dioxide, sodium carboxymethyl cellulose, and combinations thereof.

Various stabilizing agents or preservatives can be used in the present invention. The term “stabilizing agent” or “preservative” refers to any substance that can keep the prostanoid-receptor agonist chemically stable or which can slow or retard the degradation or alteration of a prostanoid-receptor agonist. For example, a stabilizing agent can protect the prostanoid-receptor agonist from instability caused by light, moisture, heat, or oxidation. In some embodiments, the stabilizing agent is lipophilic. In other embodiments, the stabilizing agent is hydrophilic. In still other embodiments, the stabilizing agent can be an antioxidant. Stabilizing agents include, but are not limited to, α-lipoic acid, α-tocopherol, ascorbyl palmitate, benzyl alcohol, biotin, bisulfites, boron, butylated hydroxyanisole (BHA), butylated hydroxytoluene (BHT), ascorbic acid and its esters, carotenoids, calcium citrate, acetyl-L-camitine, chelating agents, chondroitin, chromium, citric acid, coenzyme Q-10, cysteine, cysteine hydrochloride, 3-dehydroshikimic acid (DHS), EDTA (ethylenediaminetetraacetic acid; edetate disodium), ferrous sulfate, folic acid, fumaric acid, alkyl gallates, garlic, glucosamine, grape seed extract, gugul, magnesium, malic acid, metabisulfite, N-acetyl cysteine, niacin, nicotinomide, nettle root, ornithine, propyl gallate, pycnogenol, saw palmetto, selenium, sodium bisulfite, sodium metabisulfite, sodium sulfite, potassium sulfite, tartaric acid, thiosulfates, thioglycerol, thiosorbitol, tocopherol and their esters, e.g., tocopheral acetate, tocopherol succinate, tocotrienal, d-α-tocopherol acetate, vitamin A and its esters, vitamin B and its esters, vitamin C and its esters, vitamin D and its esters, vitamin E and its esters, e.g., vitamin E acetate, zinc, and combinations thereof.

Thus, for example, the pharmaceutical gel of the present invention can comprise: (a) a prostanoid-receptor agonist; (b) a pharmaceutically acceptable cellulose derivative, wherein the cellulose derivative is a hydroxyethyl cellulose grade having a molecular weight of about 90,000 MW, about 720,000 MW, about 1,000,000 MW, or about 1,300,000 MW; and (c) a pharmaceutically acceptable polyol, such as glycerin, wherein the ratio of the cellulose derivative to the polyol is about 1:2 to about 1:10 (w/w). The gel can further comprise an additive such as a mucoadhesive agent, for example, a cross-linked acrylic acid-based polymer.

The present invention is also directed to intravaginal dosage forms comprising a pharmaceutical gel comprising a prostanoid-receptor agonist, one or more pharmaceutically acceptable cellulose derivatives, and a pharmaceutically acceptable polyol, wherein the ratio of the cellulose derivative to the polyol is about 1:2 to about 1:10 (w/w).

The present invention is also directed to a pharmaceutical gel as described herein, and a supportive device. The term “supportive device” refers to a device suitable in size, shape and composition for placement in a vaginal tract. The device aids in providing shape and structure to the pharmaceutical gel. For example, the supportive device can be annular, such as a vaginal ring. In some embodiments, the supportive device can comprise a cavity sufficient for retaining the pharmaceutical gel.

“Intravaginal” as used herein is intended to include the local administration or application of the pharmaceutical gel of the present invention to the vaginal region, including the cervix, of a female. Exemplary intravaginal dosage forms of the present invention include, but are not limited to, a vaginal ring, a soft tablet, a capsule, a vaginal bead, a vaginal suppository, a vaginal cream and other vaginal inserts. In some aspects of the invention, the gel can be used in compression or injection molding to form a support-free gel delivery device. Such a gel matrix ring composition requires the presence a high molecular weight cellulose derivative, such as hydroxyethyl cellulose with a MW of about 720,000 or higher, to impart sufficient mechanical strength and achieve the needed balance between device viscoelasticity and desired active agent release profile. In yet other aspects, such a vaginal ring can be adapted to incorporate other core materials for mechanical reinforcement, e.g., solid rods, or for active agent release modification, e.g., hollow tubing containing different pharmaceutical compositions, gels or active agents.

The pharmaceutical gel can be directly applied to the vaginal region. In some embodiments, the gel is directly applied to the vaginal region with the use of an intravaginal applicator. In such an embodiment, the pharmaceutical gel can comprise a mucoadhesive agent to enhance the residence time of the prostanoid-receptor agonist at the site of absorption, thereby increasing the period over which the prostanoid-receptor agonist can be absorbed. Alternately, gel of the present invention can be used in conjunction with mechanical dilatation devices, such as, but not limited to, Foley's catheters, for inducing cervical ripening. In such embodiments, it is contemplated that use of the gel can be either simultaneous with, or after, the employment of the mechanical dilatation device.

In some embodiments, the intravaginal dosage form is a vaginal ring. Vaginal ring devices are well known in the art for the local administration of pharmaceutical agents to the vaginal region. For example, U.S. Pat. No. 196,979 describes a vaginal medicated-ring device. U.S. Pat. No. 3,545,439 describes an intravaginal ring-shaped device made of a solid polymer, the device containing a drug that is released by diffusion to the vagina. U.S. Pat. No. 3,920,805 describes a polymeric device with a solid, non-medicated central core and a medicated coating on the core which releases drug by diffusion. U.S. Pat. No. 4,012,496 describes a medicament-free vaginal ring having an encircling indentation with another smaller, medicament-containing ring placed in the indentation. Other vaginal medicament dispensing means are disclosed in U.S. Pat. Nos. 3,991,760; 3,924,622; 3,995,633; 4,155,991; 4,292,964; 4,286,587; 4,601,893; 4,830,860; and 5,788,977. Any such device is suitable for use with the pharmaceutical gel of the present invention.

Likewise, vaginal ring devices suitable for use with the pharmaceutical gel of the present invention can be made from polymers such as silicone elastomers; or thermoplastic elastomeric polymers such as, but not limited to, polyurethanes, copolyesters, and blends ethylene-propylene co-polymers with polypropylene. In some embodiments, the thermoplastic elastomeric material can be, but is not limited to, a thermoplastic olefin blend, an ionomer, a block copolymer, or combinations thereof. In some embodiments, the thermoplastic elastomeric material is a styrene-ethylene-butylene-styrene block copolymer, e.g., C-Flex® polymer (Consolidated Polymer Technologies, Inc., FL).

Various block copolymers can be used. In some embodiments, the block copolymer is a styrene-butadiene block copolymer. In other embodiments, the block copolymer comprises polypropylene and ethylene-propylene diene rubber. In still other embodiments, the block copolymers comprise styrene with butadiene or isoprene.

In some embodiments, the polymeric material is a thermosetting material. A thermosetting material is any material that changes irreversibly under the influence of heat from a fusible and soluble material into one which is infusible and insoluble through the formation of a covalently linked, thermally stable network. Suitable thermosetting materials include, but are not limited to, cross-linked polymers, copolymers, block copolymers, and combinations thereof.

In some embodiments of the present invention, the pharmaceutical gel of the present invention is used in combination with a vaginal ring wherein the vaginal ring comprises a cavity (or grooves) sufficient for retaining the pharmaceutical gel. In accordance with this embodiment, the gel is filled into cavities or grooves of the vaginal ring. This gel, if desired, can be heat-set by annealing the entire device at an appropriate temperature for a period of time, i.e., up to 80° C. for 5 or more minutes.

The vaginal ring of the present invention can be positioned near the cervix with the prostanoid-receptor agonist being released from one side of the device. It is to be understood that the prostanoid-receptor agonist can be released from the device from either the side facing away from the cervix or the side adjacent to the cervix. In some aspects of the invention, the vaginal ring will be positioned onto the cervix with the active agent-loaded side of the device being in contact with the cervix. In such an embodiment, the gel will undergo slight post-insertion swelling due to the local vaginal environment, this ensures that the gel component will remain in the cavities or grooves of the vaginal ring and allows for easy and complete removal of the pharmaceutical gel at a desired time interval. This characteristic reduces the amount of residual active pharmaceutical ingredient remaining in the vagina after removal of the device. Another benefit of this design is the ability to provide both immediate release or controlled release of the active agent, depending on the ratio of cellulose derivative and polyol employed in the gel. This characteristic overcomes the problem of dose-dumping associated with the vaginal use of oral tablets in administering prostanoid-receptor agonists. Additionally, the ability to completely remove the active agent greatly facilitates reversing the action of the prostanoid-receptor agonist, if desired.

In other aspects of the invention, the intravaginal dosage form can be designed to provide both a “burst release” and a “constant release” of the active agent. In such an embodiment, the intravaginal device will comprise two separate gels which, based on each gel's ratio of cellulose derivative to polyol, will provide two distinct release rates of the prostanoid-receptor agonist from a single vaginal ring device.

The vaginal rings of the present invention can further comprise an implement which allows for removal of the vaginal ring from the vaginal tract. In some aspects of the invention, the implement can be a withdrawal cord. In some aspects of the invention, the withdrawal cord can be either a wicking or non-wicking string. In some aspects, the withdrawal cord is a non-wicking string.

When employing a vaginal ring for intravaginal delivery of the pharmaceutical gel of the present invention, the vaginal ring will be capable of providing a specific release rate of the pharmaceutical gel. In some aspects of the invention, the intravaginal dosage form is a vaginal ring that provides for immediate (or “burst”) release of the active agent, e.g., misoprostol. In other aspects, the intravaginal dosage form is a vaginal ring that provides for the controlled release of the misoprostol over a specific period of time, including and up to 24 hours, or from about 1 hour to about 12 hours, or from about 2 hours to about 6 hours, at a specific release rate.

Thus, by way of non-limiting examples, the intravaginal dosage form of the present invention can be a vaginal ring, wherein about 2 μg/hour to about 100 μg/hour of a prostanoid-receptor agonist, e.g., misoprotol, is released, in some embodiments for about 1 hour to about 24 hours, or about 4 hours to about 12 hours. In yet other aspects, the intravaginal dosage form is a vaginal ring, wherein about 2 μg/hour to about 50 μg/hour of a prostanoid-receptor agonist, e.g., misoprotol, is released, in some embodiments for about 1 hour to about 24 hours, or about 4 hours to about 12 hours. In yet other aspects, the intravaginal dosage form is a vaginal ring, wherein about 2 μg/hour to about 20 μg/hour of a prostanoid-receptor agonist, e.g., misoprotol, is released, in some embodiments for about 1 hour to about 24=hours, or about 4 hours to about 12 hours.

In other aspects, the intravaginal dosage forms are directed to a vaginal ring, wherein about 12 μg/hour of a prostanoid receptor agonist, e.g., misoprotol, is released, in some embodiments for about 1 hour to about 24 hours, or about 4 hours to about 12 hours. In yet other aspects, the intravaginal dosage forms are directed to a vaginal ring, wherein about 6 μg/hour of a prostanoid receptor agonist, e.g., misoprotol, is released, in some embodiments for about 1 hour to about 24 hours, or about 4 hours to about 12 hours. In yet other aspects, the intravaginal dosage forms are directed to a vaginal ring, wherein about 3 μg/hour of a prostanoid receptor agonist, e.g., misoprotol, is released for about 1 hour to about 24 hours, or about 4 hours to about 12 hours.

The present invention is directed to a method of inducing uterine contractions in a female comprising locally administering to a vagina of the female a therapeutically effective amount of the pharmaceutical gel of the present invention. This method is intended to encompass situations wherein the female is in medical need of the inducement of uterine contractions or wherein the female voluntarily chooses to induce uterine contractions without the presence of medical necessity. Local administration into the vagina includes, but is not limited to, the use of a intravaginal dosage form such as a vaginal ring, soft tablet, capsule, vaginal bead, or vaginal suppository. Local administration also includes the application of a therapeutically effective amount of the pharmaceutical gel to the vagina of a female in need thereof using an intravaginal applicator.

The present invention is further directed to a method of inducing cervical ripening in a female comprising locally administering to a vagina of the female a therapeutically effective amount of the pharmaceutical gel of the present invention. This method is intended to encompass situations wherein the female is in medical need of cervical ripening or wherein the female voluntarily chooses to induce cervical ripening without the presence of medical necessity. Local administration into the vagina includes, but is not limited to, the use of a intravaginal dosage form such as a vaginal ring. Local administration also includes the application of a therapeutically effective amount of the pharmaceutical gel to the vagina of a female in need thereof using an intravaginal applicator.

The present invention is also directed to a method of inducing cervical dilatation in a female comprising locally administering into the vagina of the female a therapeutically effective amount of the pharmaceutical gel of the present invention. This method is intended to encompass situations wherein the female is in medical need of the inducement of cervical dilation or wherein the female voluntarily chooses to induce cervical dilation without the presence of medical necessity. The present invention is further directed to method of inducing cervical dilatation in a non-pregnant female prior to an operative and/or diagnostic procedure. Local administration into the vagina includes, but is not limited to, the use of a intravaginal dosage form such as a vaginal ring, soft tablet, capsule, vaginal bead, or a vaginal suppository. Local administration also includes the application of a therapeutically effective amount of the pharmaceutical gel to the vagina of a female in need thereof using an intravaginal applicator.

The present invention is also directed to a method of reducing and/or controlling bleeding in a female, such as post-partum hemorrhaging (PPH), which is defined as the loss of more than 500 mls of blood during a vaginal delivery or the loss of more than 1000 mls of blood during a cesarean delivery, comprising locally administering into the vagina of the female a therapeutically effective amount of the pharmaceutical gel of the present invention. The present invention is further directed to a method of controlling and/or reducing bleeding in a female in response to life threatening levels of vaginal blood loss comprising locally administering into the vagina of the female a therapeutically effective amount of the pharmaceutical gel of the present invention. Local administration into the vagina includes, but is not limited to, the use of a intravaginal dosage form such as a vaginal ring, soft tablet, capsule, vaginal bead, or a vaginal suppository. Local administration also includes the application of a therapeutically effective amount of the pharmaceutical gel to the vagina of a female in need thereof using an intravaginal applicator.

The present invention is directed to a method of inducing pre-abortion cervical priming in a female comprising locally administering into the vagina of the female a therapeutically effective amount of the pharmaceutical gel of the present invention. This method is intended to encompass situations wherein the female is in medical need of the inducement of pre-abortion cervical priming or wherein the female voluntarily chooses to induce pre-abortion cervical priming without the presence of medical necessity. In such an embodiment, the administration of the pharmaceutical gel will precede an operative and/or diagnostic procedure. Local administration into the vagina includes, but is not limited to, the use of a intravaginal dosage form such as a vaginal ring, soft tablet, capsule, vaginal bead, or a vaginal suppository. Local administration also includes the application of a therapeutically effective amount of the pharmaceutical gel to the vagina of a female in need thereof using an intravaginal applicator.

The present invention is further directed to a method of inducing a medical abortion in a female comprising locally administering into the vagina of the female a therapeutically effective amount of the pharmaceutical gel of the present invention. This method is intended to encompass situations wherein the female is in medical need of the inducement of a medical abortion or wherein the female voluntarily chooses to induce a medical abortion without the presence of medical necessity. Local administration into the vagina includes, but is not limited to, the use of a intravaginal dosage form such as a vaginal ring, soft tablet, capsule, vaginal bead, or a vaginal suppository. Local administration also includes the application of a therapeutically effective amount of the pharmaceutical gel to the vagina of a female in need thereof using an intravaginal applicator.

All of the various embodiments or options described herein can be combined in any and all variations.

The following Examples serve only to illustrate the invention, and are not to be construed in any way to limit the invention.

### EXAMPLE 1

Two different misoprostol (Everlight Chemical Industrial Corp., Taiwan) containing gel matrix formulations were prepared for use in C-flex® vaginal rings (Polymer Conversions, Inc., Orchard Park, N.Y.). Formulation Rₓ1 contained 25 μg of misoprostol dispersed in a 1:4 HEC (50% Natrosol® 250L/50% Natrosol® 250HX) (Hercules, Inc., Wilmington, Del.): glycerin (Spectrum, Inc., USA) gel matrix and was injected into a C-flex® narrow groove vaginal ring. Formulation Rₓ2 contained 50 μg of misoprostol dispersed in a 1:5 HEC (50% Natrosol® 250L/50% Natrosol® 250HX): glycerin gel matrix and was injected into a C-flex® wide groove vaginal ring. Carbopol® (BF Goodrich, Inc., Cleveland, Ohio) was added to each formulation to improve adhesion of the formulation to the groove of the respective C-flex® vaginal ring. The gel matrixes containing the misoprostol dispersions formulations in Table 1.

TABLE 1

| Components | Formulation Rₓ1 | Formulation Rₓ2 |
|---|---|---|
| Carbopol ® | 0.25 g | 0.25 g |
| Glycerin | 79.80 g | 83.125 g |
| Natrosol ® 250L | 9.975 g | 8.3125 g |
| Natrosol ® 250 HX | 9.975 g | 8.3125 g |

The C-flex® vaginal rings containing the misoprostol dispersions were prepared using the following methods.

The Carbopol® (BF Goodrich, Inc.) was dispersed in approximately one half of the total amount of glycerin with high agitation by adding small amounts of the Carbopol® over time. This was mixed until homogeneous. The mixture was heated at 45° C. until it became transparent. The HEC component was slowly added to the homogenous mixture along with the remaining glycerin. This mixture was mixed until homogenous. Then 0.13 g of misoprostol was added to 39.87 g of the prepared homogenous mix. This was further mixed in a Hauschild speed mixer until the misoprostol was fully incorporated into the matrix. The mix was transferred to 2-oz cartridges and injected into the vaginal rings. The injected vaginal rings were annealed in an oven at 80° C. for 30 min.

### EXAMPLE 2

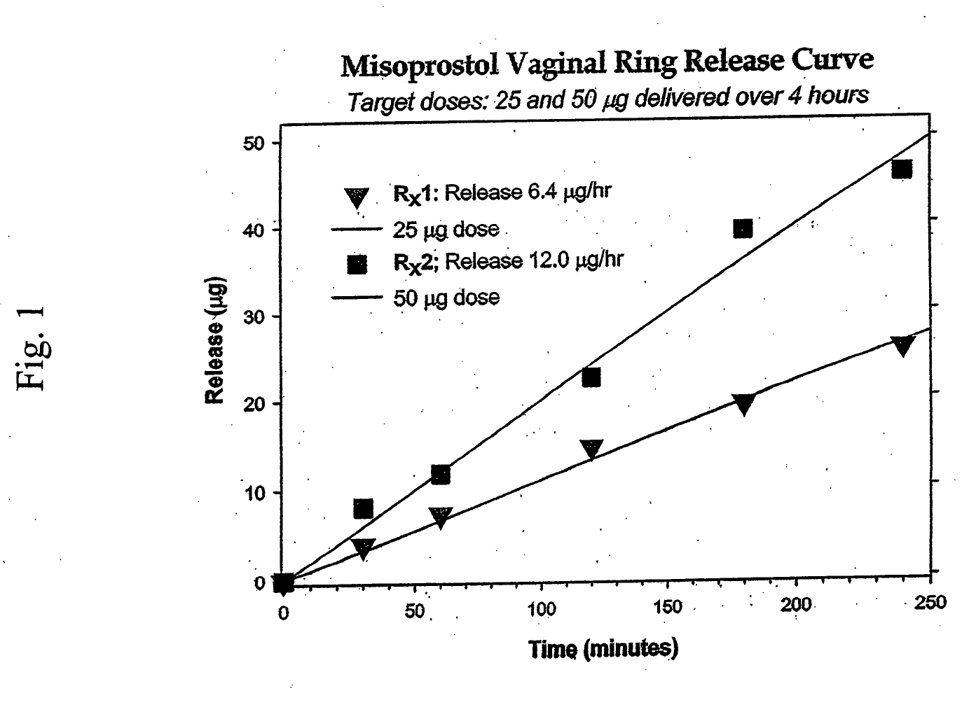

The vaginal rings prepared according to the methods described in Example 1 were tested to determine their in vitro release profiles. The release rates of misoprostol from the different vaginal ring devices of Example 1 were analyzed using a Modified USP Apparatus 2 with paddles. The vaginal rings were placed in 350 mL of deionized water for 4 hours at 50 RPM. Results (misoprostol released (μg/hr)) for vaginal rings containing Formulations Rₓ1 and Rₓ2 are found in FIG. 1.

### EXAMPLE 3

Three different misoprostol (Everlight Chemical Industrial Corp.) containing gel matrixes were prepared for use in C-flex® narrow groove vaginal rings (Polymer Conversions, Inc.). Formulation 1 contained 50 μg of misoprostol dispersed in a 1:4 HEC (Natrosol® 250M) (Hercules, Inc.): glycerin (EM Science, Gibbstown, N.J.) gel matrix. Noveon® AA1 (Noveon, Inc., Cleveland, Ohio) was not added to this formulation. Formulation 2 contained 50 μg of misoprostol dispersed in a 1:5 HEC (Natrosol® 250HX): glycerin gel matrix. Noveon® AA1 was added to Formulation 2 to improve adhesion of the formulation to the groove of the C-flex® vaginal ring. Formulation 3 contained 50 μg of misoprostol dispersed in a 1:5 HEC (80% Natrosol® 250HX/20% Natrosol® 250L): glycerin gel matrix. Noveon® AA1 was added to Formulation 3 to improve adhesion of the formulation to the groove of the C-flex® vaginal ring. The gel matrixes containing the misoprostol dispersions had the following formulations in Table 2.

TABLE 2

| Components | Formulation 1 | Formulation 2 | Formulation 3 |
|---|---|---|---|
| Noveon ® AA1 | | 0.25 g | 0.25 g |
| Glycerin | 31.89 g | 83.125 g | 83.125 g |
| Natrosol ® 250 L | | | 3.325 g |
| Natrosol ® 250 HX | | 16.625 g | 13.30 g |
| Natrosol ® 250 M | 7.940 g | | |

The C-flex® narrow groove vaginal rings containing the misoprostol dispersions were prepared using the following methods.

Formulation 1 was prepared by weighing 0.14 g of misoprostol, adding 7.970 g of HEC (Natrosol® 250 M), followed by the addition of 31.89 g of glycerin. This blend was mixed twice (2×) for 26 seconds (or until smooth) in a Hauschild speed mixer, with hand mixing in-between. This misoprostol dispersion was dispensed into the groove of the C-flex® narrow groove vaginal ring. The filled ring was annealed in the oven at 80° C. for 30 min. Formulation 2 was prepared by dispersing the Noveon® AA1 in approximately one half of the glycerin component with high agitation by adding small amounts of the carbopol over time with high agitation. This was mixed until homogeneous. The mixture was heated at 45° C. until it became transparent. The HEC component (Natrosol® 250HX) was slowly added to the homogenous mixture along with the remaining glycerin. This was mixed until homogenous. Then 0.14 g of misoprostol was added to 39.86 g of the prepared mix. This was mixed in a Hauschild speed mixer until the misoprostol was fully incorporated into the matrix. The mix was transferred to 2-oz cartridges and injected into the vaginal ring. The injected vaginal ring was annealed in an oven at 80° C. for 30 min. Formulation 3 was prepared by dispersing the Noveon® AA1 in approximately one half of the glycerin component with high agitation by adding small amounts of the carbopol over time with high agitation. This was mixed until homogeneous. The mixture was heated at 45° C. until it became transparent. The HEC component (80% Natrosol® 250HX/20% Natrosol® 250L) was slowly added to the homogenous mixture along with the remaining glycerin. This was mixed until homogenous. Then 0.14 g of misoprostol was added to 39.86 g of the prepared mix. This was mixed in a Hauschild speed mixer until the misoprostol was fully incorporated into the matrix. The mix was transferred to 2-oz cartridges and injected into the vaginal ring. The injected vaginal ring was annealed in an oven at 80° C. for 30 min.

### EXAMPLE 4

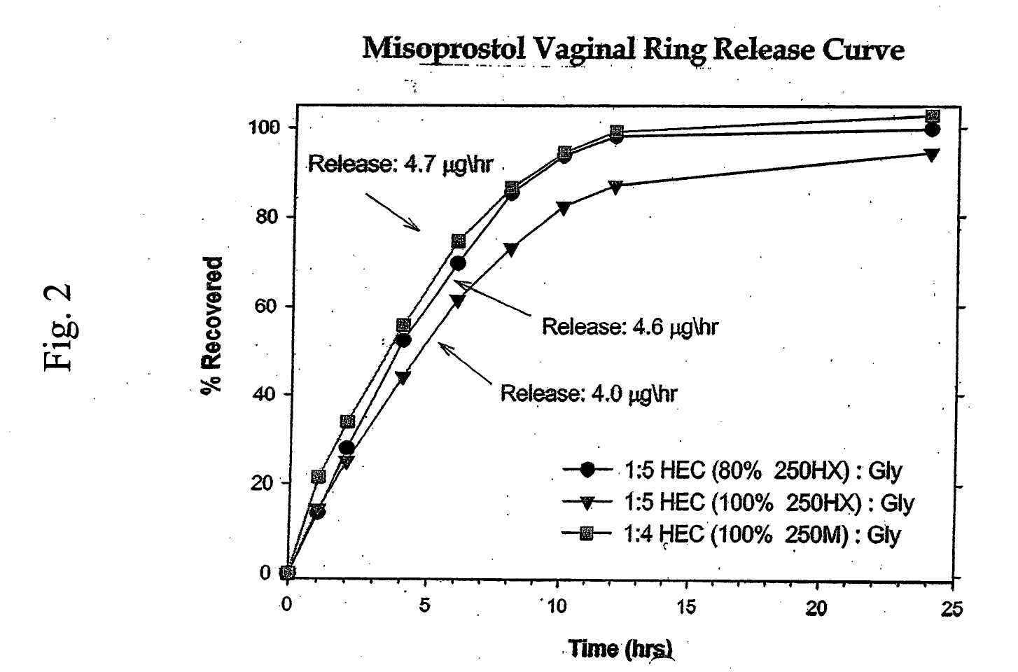

The vaginal rings prepared according to the methods described in Example 3 were tested to determine their in vitro release profiles. The release rate of misoprostol from the different vaginal ring devices of Example 3 were analyzed using a Modified USP Apparatus 2 with paddles. The vaginal rings were placed in 350 mL of deionized water for 24 hours at 50 RPM. Results (misoprostol released (μg/hr)) for vaginal rings containing Formulations 1-3 are found in FIG. 2.

### EXAMPLE 5

Three different misoprostol (Everlight Chemical Industrial Corp.) containing gel matrixes were prepared for use in C-flex® groove-containing vaginal rings (Polymer Conversions, Inc.). Formulation 1 (Low Dose) contained 12.5 μg of misoprostol dispersed in a 1:6 HEC (75% Natrosol® 250L/25% Natrosol® 250HX) (Hercules, Inc.): glycerin (EM Science, Gibbstown, N.J., USA) gel matrix. Formulation 2 (Medium Dose) contained 25 μg of misoprostol dispersed in a 1:6 HEC (75% Natrosol® 250L/25% Natrosol® 250HX): glycerin gel matrix. Formulation 3 (High Dose) contained 50 μg of misoprostol dispersed in a 1:6 HEC (75% Natrosol® 250L/25% Natrosol® 250HX): glycerin gel matrix. The gel matrixes containing the misoprostol dispersions had the formulations in Table 3.

TABLE 3

| | Ring Composition | | | | | |
|---|---|---|---|---|---|---|
| | Formulation 1 | | Formulation 2 | | Formulation 3 | |
| | 12.5 μg/day | | 25 μg/day | | 50 μg/day | |
| Ingredients | g | % | g | % | g | % |
| Misoprostol | 0.0015 | 0.08 | 0.003 | 0.17 | 0.006 | 0.33 |
| Glycerin, USP | 1.54 | 85.64 | 1.54 | 85.57 | 1.54 | 85.43 |
| Natrosol ® 250 L | 0.19 | 10.71 | 0.19 | 10.7 | 0.19 | 10.68 |
| Natrosol ® 250 HX | 0.06 | 3.57 | 0.06 | 3.56 | 0.06 | 3.56 |

The C-flex® groove-containing rings containing the misoprostol dispersions were prepared using the following methods.

Formulations 1-3 were prepared by weighing the misoprostol component and then adding the HEC component (75% Natrosol® 250L/25% Natrosol® 250HX). This was followed by the addition of the glycerin component. This blend was mixed twice until smooth in a Hauschild speed mixer. The misoprostol dispersions were then dispensed into the groove of the C-flex® groove-containing vaginal rings. The filled rings were annealed in the oven at 80° C. for 30 min.

### EXAMPLE 6

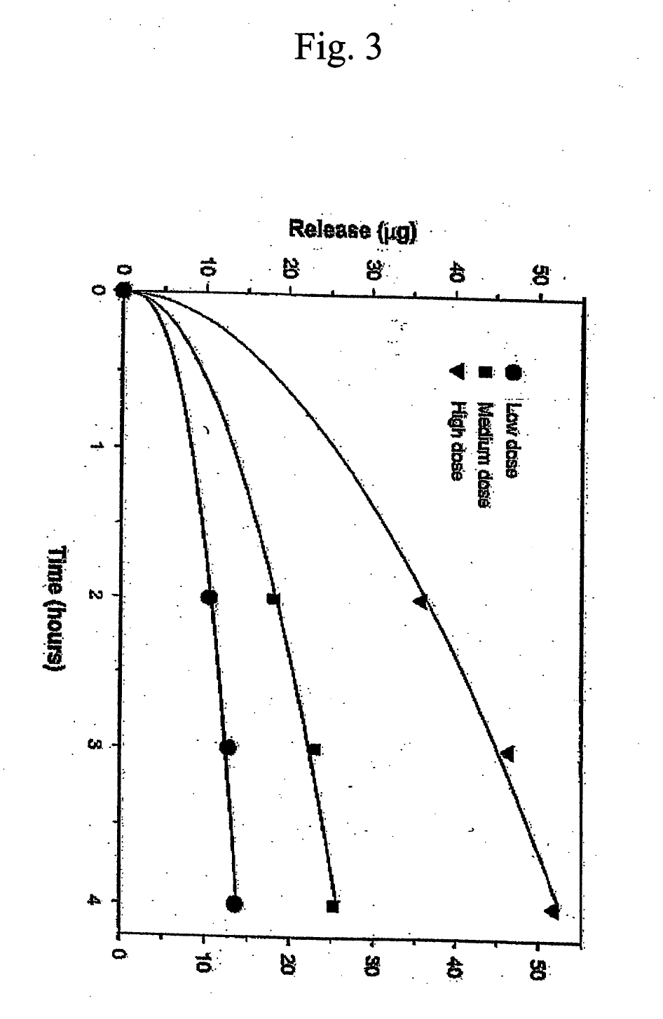

The vaginal rings prepared according to the methods described in Example 5 were tested to determine their in vitro release profiles. The release rate of misoprostol from the different vaginal ring devices of Example 5 were analyzed using a Modified USP Apparatus 2. The vaginal rings were placed in 75 mL of acetate buffer (pH=4.5) for 4 hours. The in vitro release profiles are found in FIG. 3. Formulation 1 (Low Dose) (-●-) displayed an overall release rate of misoprostol of 4.16 μg/hour. Formulation 2 (Medium Dose) (-▪-) displayed an overall release rate of misoprostol of 6.25 μg/hour. Formulation 3 (High Dose) (-▾-) displayed an overall release rate of misoprostol of 12.5 μg/hour.

### EXAMPLE 7

A misoprostol (Everlight Chemical Industrial Corp.) gel matrix was prepared for use in 30 C-flex® VR (Polymer Conversions, Inc.) vaginal rings having a single continuous groove (channel) of 6.5 mm. The formulation contained 60 μg of misoprostol dispersed in a 1:6 HEC (75% Natrosol® 250L/25% Natrosol® 250HX) (Hercules, Inc.):propylene glycol (Spectrum, Inc.) gel matrix. The gel matrix containing the misoprostol had the formulation in Table 4.

TABLE 4

| Component | Amount Required (g) |
|---|---|
| Natrosol ® 250L | 10.679 |
| Natrosol ® 250HX | 3.560 |
| Propylene Glycol | 85.4 |
| Misoprostol | 0.333 |

The C-flex® VR vaginal rings containing the misoprostol matrix were prepared using the following methods.

The formulation was prepared by initially weighing the misoprostol dispersion in a mixing cup. Next, the HEC components were separately weighed. The weighed HEC components were then added into the mixing cup containing the weighed misoprostol dispersion. Finally, the propylene glycol was added. The blend was mixed three times (3×) for 25 seconds at 3000 RPM in a Hauschild speed mixer, with hand mixing in-between. The misoprostol matrix was then dispensed into the groove of the C-flex® VR vaginal rings. The filled rings were cured in the oven at 50° C. for 60 min. The rings were removed from the oven and allowed to cool for 10-15 min.

### EXAMPLE 8

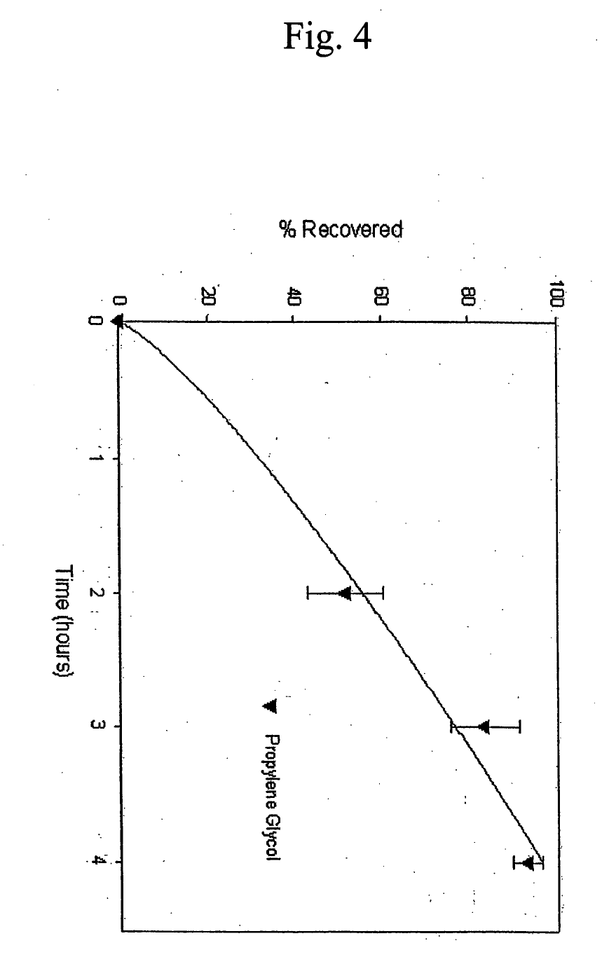

The vaginal rings prepared according to the method described in Example 7 were tested to determine their in vitro release profiles. The release rate of misoprostol from the vaginal ring devices of Example 7 were analyzed using a Modified USP Apparatus 2. The vaginal rings were placed in 75 mls of acetate buffer (pH=4.5) for 4 hours. The results (misoprostol released (μg/hr)) for the vaginal rings containing the formulation of Example 7 are found in FIG. 4.

### EXAMPLE 9

A misoprostol (Everlight Chemical Industrial Corp.) gel matrix was prepared for use in 10 C-flex® VR (Polymer Conversions, Inc.) vaginal rings having a single continuous groove (channel) of 6.5 mm. The formulation contained 60 μg misoprostol dispersed in a 1:6 HEC (75% Natrosol® 250L/25% Natrosol® 250HX) (Hercules, Inc.):propylene glycol (Spectrum, Inc., USA)+PEG-400 30% (Spectrum, Inc., USA)+glycerin (EM Science, USA) gel matrix. The gel matrix containing the misoprostol had the formulation in Table 5.

TABLE 5

| Components | Amount Required (g) |
|---|---|
| Natrosol ® 250L | 4.272 |
| Natrosol ® 250HX | 1.424 |
| Propylene Glycol | 10.82 |
| PEG-400 | 10.248 |
| Glycerin | 13.09 |
| Misoprostol | 0.133 |

The C-flex® VR vaginal rings containing the misoprostol matrix were prepared using the following methods.

The formulation was prepared by initially weighing the misoprostol dispersion in a mixing cup. Next, the HEC components were separately weighed. The weighed HEC components were then added into the mixing cup containing the weighed misoprostol dispersion. The propylene glycol, PEG-400 and glycerin were then added. The blend was mixed three times (3×) for 26 seconds at 3000 RPM in a Hauschild speed mixer, with hand mixing in-between. The misoprostol matrix was then dispensed into the groove of the C-flex® VR vaginal rings. The filled rings were cured in the oven at 50° C. for 30 min. The rings were removed from the oven and allowed to cool to room temperature for 5 min.

### EXAMPLE 10

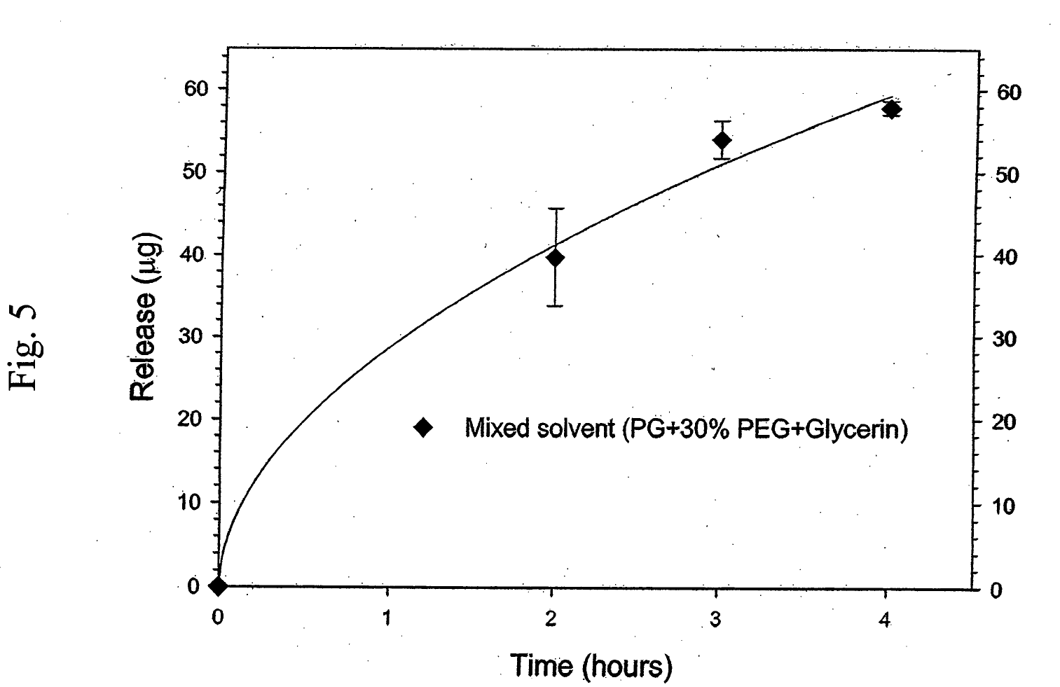

The vaginal rings prepared according to the method described in Example 9 were tested to determine their in vitro release profiles. The release rate of misoprostol from the vaginal ring devices of Example 9 were analyzed using a Modified USP Apparatus 2. The vaginal rings were placed in 75 mL of acetate buffer (pH=4.5) for 4 hours. The results (misoprostol released (μg/hr)) for the vaginal rings containing the formulation of Example 9 are found in FIG. 5.

### EXAMPLE 11

A misoprostol (Everlight Chemical Industrial Corp.) gel matrix was prepared for use in 10 C-flex® VR (Polymer Conversions, Inc.) vaginal rings having a single continuous groove (channel) of 6.5 mm. The formulation contained 60 μg misoprostol dispersed in a 1:6 HEC (75% Natrosol® 250L/25% Natrosol® 250HX) (Hercules, Inc.):propylene glycol (Spectrum, Inc., USA)+1% glycerin (EM Science, USA) gel matrix. The gel matrix containing the misoprostol had the formulation in Table 6.

TABLE 6

| | 1% Glycerin |
|---|---|
| Components | Amount Required (g) |
| Natrosol ® 250L | 4.272 |
| Natrosol ® 250HX | 1.424 |
| Propylene Glycol | 33.82 |
| Glycerin | 0.34 |
| Misoprostol | 0.133 |

The C-flex® VR vaginal rings containing the misoprostol matrix were prepared using the following methods.

The formulation was prepared by initially weighing the misoprostol dispersion in a mixing cup. Next, the HEC components were separately weighed. The weighed HEC components were then added into the mixing cup containing the weighed misoprostol dispersion. The propylene glycol and glycerin were then added. The blend was mixed three times (3×) for 26 seconds at 3000 RPM in a Hauschild speed mixer, with hand mixing in-between. The misoprostol matrix was then dispensed into the groove of the C-flex® VR vaginal rings. The filled rings were cured in the oven at 50° C. for 30 min. The rings were removed from the oven and allowed to cool to room temperature for 5 min.

### EXAMPLE 12

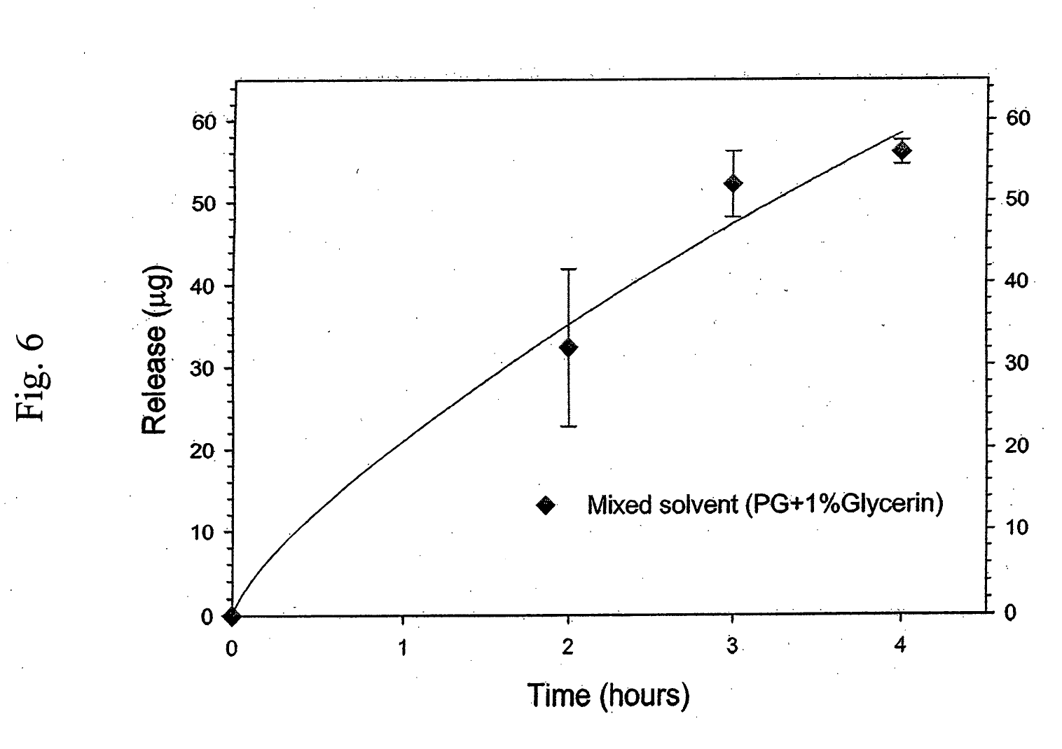

The vaginal rings prepared according to the method described in Example 11 were tested to determine their in vitro release profiles. The release rate of misoprostol from the vaginal ring devices of Example 11 were analyzed using a Modified USP Apparatus 2. The vaginal rings were placed in 75 mL of acetate buffer (pH=4.5) for 4 hours. The results (misoprostol released (μg/hr)) for the vaginal rings containing the formulation of Example 11 are found in FIG. 6.

These examples illustrate possible formulations of the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patents applications, issued or foreign patents, or any other documents, are each entirely incorporated by reference herein, including all data, tables, figures, and text presented in the cited documents.

What is claimed:

1. A controlled release pharmaceutical gel for vaginal administration, the pharmaceutical gel comprising:
(a) misoprostol;
(b) a cellulose derivative; and
(c) a polyol;
wherein the gel is a substantially nonaqueous gel, and wherein the gel forms a hydrogel when placed in a vaginal tract.

2. The pharmaceutical gel of claim 1, further comprising (d) a mucoadhesive agent.

3. The pharmaceutical gel of claim 2, wherein the mucoadhesive agent is selected from the group consisting of a cross-linked acrylic acid-based polymer, polycarbophil, chitosan, polyethylene oxide, and combinations thereof.

4. The pharmaceutical gel of claim 2, wherein the mucoadhesive agent is a cross-linked acrylic acid-based polymer.

5. The pharmaceutical gel of claim 1, wherein the cellulose derivative is selected from the group consisting of methyl cellulose, hydroxyethyl cellulose, hydroxypropyl cellulose, hydroxypropyl methylcellulose, and combinations thereof.

6. The pharmaceutical gel of claim 5, wherein the cellulose derivative is hydroxyethyl cellulose having a molecular weight from about 50,000 MW to about 2,000,000 MW.

7. The pharmaceutical gel of claim 6, wherein about 75% (w/w) or greater of the cellulose derivative is about 80,000 MW to about 100,000 MW and about 25% (w/w) or less of the cellulose derivative is about 800,000 MW to about 1,200,000 MW.

8. The pharmaceutical gel of claim 6, wherein about 50% (w/w) of the cellulose derivative is about 80,000 MW to about 100,000 MW and about 50% (w/w) of the cellulose derivative is about 800,000 MW to about 1,200,000 MW.

9. The pharmaceutical gel of claim 6, wherein about 20% (w/w) or less of the cellulose derivative is about 80,000 MW to about 100,000 MW and about 80% (w/w) or greater of the cellulose derivative is about 800,000 MW to about 1,200,000 MW.

10. The pharmaceutical gel of claim 1, wherein the ratio of the cellulose derivative to polyol is about 1:2 to about 1:10 (w/w).

11. The pharmaceutical gel of claim 10, wherein the ratio of the cellulose derivative to polyol is about 1:4 to about 1:7 (w/w).

12. The pharmaceutical gel of claim 1, wherein the polyol is glycerin, propylene glycol, polyethylene glycol, or combinations thereof.

13. The pharmaceutical gel of claim 12, wherein the polyol is glycerin.

14. The pharmaceutical gel of claim 1, further comprising a pharmaceutically acceptable additive, pharmaceutically acceptable stabilizing agent, or combination thereof.

15. The pharmaceutical gel of claim 14, wherein the pharmaceutically acceptable additive is selected from the group consisting of polaxomers, carbomers, polyvinyl alcohol, silicon dioxide, sodium carboxymethyl cellulose, and combinations thereof.

16. The pharmaceutical gel of claim 14, wherein the pharmaceutically acceptable stabilizing agent selected from the group consisting of α-lipoic acid, α-tocopherol, ascorbyl palmitate, benzyl alcohol, biotin, bisulfites, boron, butylated hydroxyanisole, butylated hydroxytoluene, ascorbic acid, carotenoids, calcium citrate, acetyl-L-carnitine, chelating agents, chondroitin, chromium, citric acid, coenzyme Q-10, cysteine, cysteine hydrochloride, 3-dehydroshikimic acid, EDTA, ferrous sulfate, folic acid, fumaric acid, alkyl gallates, garlic, glucosamine, grape seed extract, gugul, magnesium, malic acid, metabisulfite, N-acetyl cysteine, niacin, nicotinomide, nettle root, ornithine, propyl gallate, pycnogenol, saw palmetto, selenium, sodium bisulfite, sodium metabisulfite, sodium sulfite, potassium sulfite, tartaric acid, thiosulfates, thioglycerol, thiosorbitol, tocopherol, tocopherol acetate, tocopherol succinate, tocotrienal, d-α-tocopherol acetate, vitamin A, vitamin B, vitamin C, vitamin D, vitamin E, zinc and combinations thereof.

17. The pharmaceutical gel of claim 1, further comprising a supportive device.

18. The pharmaceutical gel of claim 17, wherein the supportive device is annular.

19. The pharmaceutical gel of claim 18, wherein the supportive device comprises a cavity sufficient for retaining the pharmaceutical gel.

20. An intravaginal dosage form comprising the pharmaceutical gel of claim 1, wherein the dosage form is selected from the group consisting of a vaginal ring, a soft tablet, a capsule, a vaginal bead, and a vaginal suppository.

21. The intravaginal dosage form of claim 20, further comprising a withdrawal cord.

22. The intravaginal dosage form of claim 20, wherein the dosage form is a vaginal ring.

23. The intravaginal dosage form of claim 22, wherein the vaginal ring comprises a cavity sufficient for retaining the pharmaceutical gel.

24. The intravaginal dosage form of claim 22, wherein the vaginal ring is suitable for immediate release of the misoprostol.

25. The intravaginal dosage form of claim 22, wherein the vaginal ring is suitable for controlled release of the misoprostol.

26. The intravaginal dosage form of claim 25, wherein the vaginal ring has a release rate of about 2 μg/hour to about 100 μg/hour of misoprostol.

27. The intravaginal dosage form of claim 25, wherein the vaginal ring has a release rate of about 2 μg/hour to about 50 μg/hour of misoprostol.

28. The intravaginal dosage form of claim 25, wherein the vaginal ring has a release rate of about 2 μg/hour to about 20 μg/hour of misoprostol.

29. The intravaginal dosage form of claim 25, wherein the misoprostol is released for about 1 hour to about 24 hours.

30. A pharmaceutical gel comprising:
(a) misoprostol;
(b) two or more cellulose derivatives;
(c) a polyol; and
(d) a mucoadhesive agent;
wherein the gel is a substantially nonaqueous gel, and wherein the gel forms a hydrogel when placed in a vaginal tract.

31. A method of inducing uterine contractions in a female, the method comprising locally administering to the vagina of the female a therapeutically effective amount of the pharmaceutical gel of claim 1.

32. The method of claim 31, wherein the local administration to the vagina is achieved by using an intravaginal applicator.

33. A method of inducing cervical ripening in a female, the method comprising locally administering to a vagina of the female a therapeutically effective amount of the pharmaceutical gel of claim 1.

34. The method of claim 33, wherein the local administration to the vagina is achieved by using a vaginal ring, a soft tablet, a capsule, a vaginal bead, or a vaginal suppository.

35. A method of inducing cervical dilatation in a female, the method comprising locally administering to the vagina of the female a therapeutically effective amount of the pharmaceutical gel of claim 1.

36. The method of claim 35, wherein the local administration to the vagina is achieved by using a vaginal ring, a soft tablet, a capsule, a vaginal bead, or a vaginal suppository.
